(19) United States
(12) Plant Patent Application Publication
(10) Pub. No.: US 2003/0145358 P1
(43) Pub. Date: Jul. 31, 2003

(54) Magnolia intergeneric hybrid named Katie-O Early

(76) Inventor: S. Christopher Early, Atlanta, GA (US)

Correspondence Address: S CHRISTOPHER EARLY, 4341 HARRIS VALLEY RD, ATLANTA, GA, 30327, US

(21) Appl. No.: 10/059,400

(22) Filed: Jan. 31, 2002

(51) Int. Cl. A01H 5/00

(52) U.S. Cl. PLT/223

(57) ABSTRACT

An intergeneric hybrid magnolia named Katie-O Early, particularly characterized by having pink flowers on an evergreen tree.

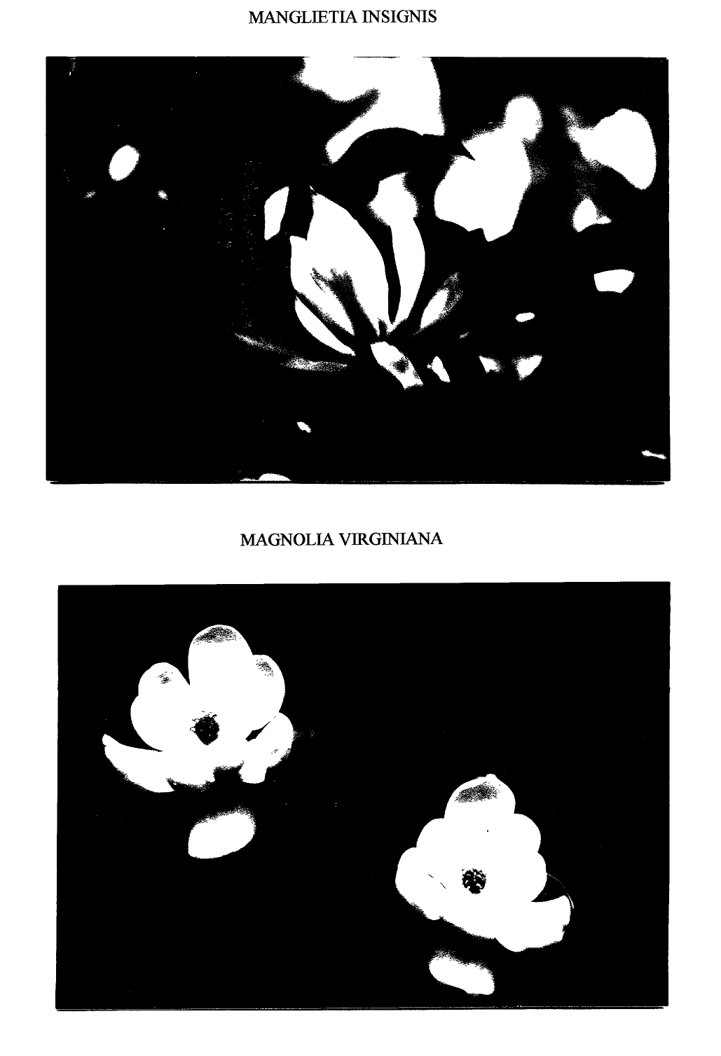

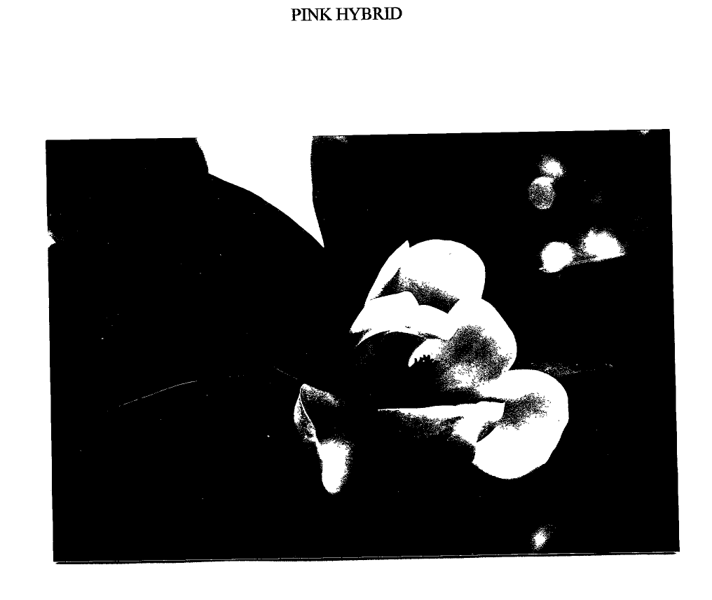

### BACKGROUND OF A NEW PLANT

This invention relates to a new and distinct intergeneric hybrid of Manglietia insignis and Magnolia virginiana, both members of the magnolia family, Magnoliaceae. The distinct features of this hybrid are it's pink flowers on an evergreen plant.

The Manglietia insignis, from China, was secured as a cutting from the Strybing Arboretum in San Francisco over thirty years ago. The rooted cutting was subsequently planted in the atrium (enclosed garden) of the home of S. Christopher Early in Atlanta.

The Magnolia virginiana is a version of this species growing around Savannah, Ga. which blooms as a small plant. It's seedlings are often cut down by mowers clearing the roadside. Several of these seedlings were planted near the home of S. Christopher Early in Atlanta.

After several years the M. insignis bloomed and pollen from the M. virginiana was applied to the pistils of the M. insignis flowers over a two year period. A fertile seed pod subsequently appeared with several seed.. From these seed have come six seedlings, one of which has bloomed and is the subject of this application. It and one of the other seedlings are over twelve feet tall. All of the siblings have survived cold spells over twenty five years down to as low as ten degrees F. The size of the bloom are between those of the two parents, about 12 cm. The seed pods from this plant do not form pollen nor do the pistils accept pollen but dry up after a week or so. This condition could change with more favorable growing conditions. Cuttings have been rooted and grafts have been made.

I have named the flower of this sibling , Katie-O Early, after my mother who was a great lover of people and flowers.

I claim:

1. A new an distinct intergeneric hybrid of a pink flowered evergreen magnolia named, Katie-O Early, as illustrated and described.
